(19) United States
(12) Patent Application Publication
(10) Pub. No.: US 2007/0260165 A1
(43) Pub. Date: Nov. 8, 2007

(54) Combined Bandage and Fastener System

(76) Inventor: Ross Johnson, Anderson, SC (US)

Correspondence Address: CAHN & SAMUELS LLP, 2000 P STREET NW, SUITE 200, WASHINGTON, DC, 20036, US

(21) Appl. No.: 11/745,184

(22) Filed: May 7, 2007

Related U.S. Application Data
(60) Provisional application No. 60/746,539, filed on May 5, 2006.
(60) Provisional application No. 60/822,198, filed on Aug. 11, 2006.

(51) Int. Cl. A61F 15/00; A61F 13/00

(52) U.S. Cl. 602/41; 602/48

(57) ABSTRACT

A combined bandage and fastener system includes a flexible bandage having a first end and a second end; at least one end brake attached to a surface of said flexible bandage and disposed proximate to the first end; and a plurality of brakes attached to at least one surface of the flexible bandage between the first end and the second end of the bandage. Each brake includes a strip of a dense arrangement of hooks.

### I. Cross Reference to Related Applications

This application claims the benefit of U.S. Provisional Patent Application No. 60/746,539, filed May 5, 2006, which is incorporated herein by reference.

This application claims the benefit of U.S. Provisional Patent Application No. 60/822,198, filed Aug. 11, 2006, which is incorporated herein by reference.

### II. Field of the Invention

The present invention relates to a bandage for the care and treatment of wounds. More particularly, the present invention relates to a combined bandage and fastener system in which a bandage has a plurality of fasteners or brakes attached thereto, thereby preventing unwanted unraveling of the bandage while also allowing for quick and effective use of the bandage to treat a wide array of wounds.

### III. BACKGROUND OF THE INVENTION

One of the leading causes of death on military battlegrounds and in hospital trauma units is severe blood loss and the associated shock that it produces. Therefore, one of the most vital priorities of caregivers (medics, first responders, and medical providers) is to control and stop bleeding. There are many devices and methods that may be employed to control bleeding. One of the most commonly used devices for bleeding control is the elastic bandage.

Elastic bandages have been known for many years. These bandages are sometimes called “roller” bandages or “wraps” and come in rolls having various fasteners. Such bandages are sold under brand names such as ACE® wrap.

Elastic bandages are a preferred wound dressing by caregivers ranging from physicians and trained medics to individual first responders with minimal first aid training. Elastics bandages are highly adaptable to treat a range of wounds and conform to virtually any body part. Elastic bandages provide many benefits at the wound site, including applying varying degrees of compression and support to the wound, as well as providing a sterile barrier around the wound.

However, most elastic bandages are difficult to handle because they unroll uncontrollably when ready for use and the fastener is applied to an end of the bandage. This configuration is particularly problematic because the time spent on re-rolling the bandage and gaining control of it increases the risk of blood loss and associated complications. Thus, a need still exists for a bandage which is easy to control and for a method for treating wounds quickly and effectively while reducing the risk of contamination and infection.

### IV. SUMMARY OF THE INVENTION

According to an aspect of the present invention, a combined bandage and fastener system is provided comprising a flexible bandage having a first end and a second end; at least one end brake attached to a surface of the flexible bandage and disposed proximate to at least one of the first end or the second end of the bandage; and a plurality of brakes attached to at least one surface of the flexible bandage between the first end and the second end of the bandage. Each brake of the plurality of brakes comprises a strip of a dense arrangement of hooks.

According to another aspect of the invention, a combined bandage and fastener system is provided comprising an elastic bandage; a plurality of brakes comprising strips of a dense arrangement of hooks, the strips spaced apart on a first surface of the elastic bandage and extending the width of the elastic bandage; and at least one end fastener disposed at an end of the elastic bandage.

According to another aspect of the invention, a method for packaging a combined bandage and fastener system is provided comprising extending the bandage lengthwise; folding the second end of the bandage; rolling the bandage from the second end towards the first end thus forming a rolled body; and attaching the at least one end brake to the rolled body.

According to an aspect of the invention, a method of applying the combined bandage and fastener system is provided comprising unrolling the bandage a user-selected amount to one of the plurality of brakes; applying the bandage to a wound; wrapping the wound with the bandage; and securing the bandage with at least one of the plurality of brakes or the at least one end brake.

According to another aspect of the invention, a wound dressing kit is provided comprising a sealed receptacle comprising a combined bandage and fastener system and a wound pad.

### V. BRIEF DESCRIPTION OF THE DRAWINGS

The present invention is described with reference to the accompanying drawings. In the drawings, like reference numbers indicate identical or functionally similar elements.

FIG. 1 illustrates a perspective view of the combined bandage and fastener system according to an embodiment of the present invention.

FIG. 2 illustrates a perspective view of the combined bandage and fastener system of FIG. 1 with a wound pad.

FIG. 3 is a schematic diagram of the combined bandage and fastener system of the present invention.

FIG. 4 is a schematic diagram of a partially-rolled combined bandage and fastener system of the present invention.

FIG. 5 illustrates a kit comprising a combined bandage and fastener system according to the present invention.

Given the following enabling description of the drawings, the apparatus should become evident to a person of ordinary skill in the art.

### VI. DETAILED DESCRIPTION OF THE DRAWINGS

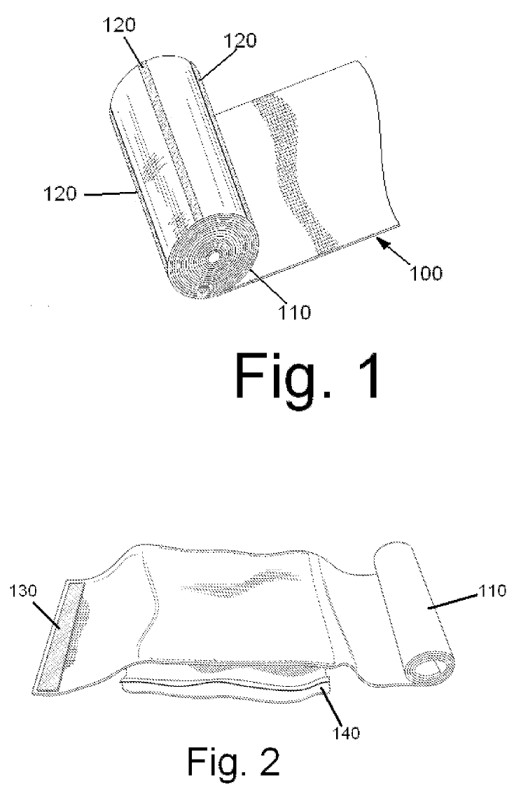

FIGS. 1-5 illustrate a combined bandage and fastener system comprising a bandage having a plurality of fasteners or brakes attached thereto, thereby preventing unwanted unraveling of the bandage while also allowing for quick and effective use of the bandage to treat a wide array of wounds.

By reducing the risk of accidental unrolling of the bandage, the following risks are minimized: (1) contaminating a clean bandage; (2) waste of a bandage due to contamination; (3); lost time while managing loose ends; and (4) loss of pressure after applying the bandage due to shifting of material. The plurality of fasteners or brakes also provides grip surfaces during the wrapping process and assist in maintaining the desired pressure and position of the bandage. This is extremely helpful when changing angles during the wrapping process or if bandaging awkward injuries.

In this detailed description, references to “one embodiment”, “an embodiment”, or “in embodiments” mean that the feature being referred to is included in at least one embodiment of the invention. Moreover, separate references to “one embodiment”, “an embodiment”, or “in embodiments” do not necessarily refer to the same embodiment; however, neither are such embodiments mutually exclusive, unless so stated, and except as will be readily apparent to those skilled in the art. Thus, the invention can include any variety of combinations and/or integrations of the embodiments described herein.

As used herein “substantially” or “about” are relative modifiers intended to indicate permissible variation from the characteristic so modified. It is not intended to be limited to the absolute value or characteristic which it modifies but rather approaching or approximating such a physical or functional characteristic.

As illustrated in FIG. 1, a combined bandage and fastener system 100 according to the present invention comprises a bandage 110 having a first end and a second end and a length longer than its width. For example, the width may be about 4 inches and the length may be about 72 inches. In embodiments, the bandage is made from a flexible or elastic material. In general, the bandage material is sufficiently porous to allow for the transmission of air and moisture vapor through the material. In addition, materials that may be sterilized, including radiation sterilized, are preferred. A particularly preferred bandage is the ACE® bandage available from Becton, Dickinson and Company of Franklin Lakes, N.J.

The bandage 110 has a plurality of fasteners or brakes 120 attached to at least one surface of the bandage between the first end and the second end of the bandage. The brakes 120 are placed over at least a portion of the bandage. In accordance with the invention, brakes 120 may be disposed on a top surface, a bottom surface, or both surfaces of bandage 110. Preferably, brakes 120 are fixedly attached to bandage 110, for example, sewn to the bandage. Alternatively, brakes 120 may be snap fit to the bandage 110 or affixed thereto by an adhesive.

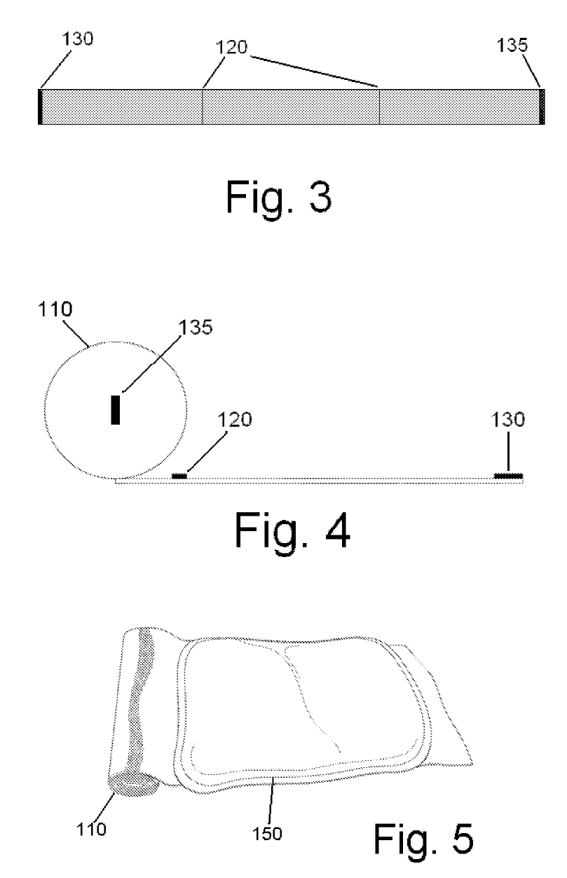

The bandage 110 may have at least one end brake attached to a surface of the bandage and disposed proximate to at least one of the first end or the second end of bandage 110. As illustrated in FIG. 2 and FIG. 3, bandage 110 may have first and second end brakes 130, 135 disposed at first and second ends of the bandage 110, respectively. In embodiments, a first end brake 130 is attached to a first surface (e.g., top surface) disposed proximate a first end of the bandage, and the second end brake 135 is attached to a second surface (e.g., bottom surface) disposed proximate a second end of the bandage. Preferably, end brakes 130, 135 are fixedly attached to bandage 110, for example, sewn to the bandage. Alternatively, end brakes 130, 135 may be snap fit to the bandage 110 or affixed thereto by an adhesive. Brakes 120 are disposed between the end brakes 130, 135. Brakes 120 and end brakes 130, 135 minimize the unintentional unraveling of the bandage 110 during application. The brakes also facilitate securing the bandage during final packaging.

As illustrated in FIG. 2, the surface of the bandage 110 may include a removably affixable wound pad 140. In embodiments, the wound pad 140 may be fixedly attached to the bandage 110, for example, sewn into the bandage.

As illustrated in FIG. 1 and FIG. 3, brakes 120 and the at least one end brake (e.g., 130, 135) may be disposed on or attached to a surface of bandage 110 in the form of strips extending the width of the bandage. Alternatively, the brakes may be in the form of strips extending along part of the length of the bandage or disposed at an angle to a lateral axis of the bandage. Brakes 120 are preferably evenly spaced along the length of bandage 110. For example, each brake 120 may be evenly spaced from adjacent brakes by any suitable distance, up to several inches. However, in some embodiments, the distance between brakes may vary.

As illustrated in FIG. 3, a brake 120 may be spaced from a first end of the bandage 110 by a distance equal to about one third (⅓) of the length of the bandage. A second brake 120 may be spaced from the first end of the bandage 110 by a distance equal to about two thirds (⅔) of the length of the bandage. In embodiments, the at least one end brake may have a width wider than the plurality of brakes 120. For example, the width of brakes 120 may be about one quarter (¼) of an inch and the width of the end brakes 130, 135 may be about one inch.

The brakes comprise structural elements that are capable of adhering to the bandage. Preferably, the brakes 120 and the at least one end brake (e.g., brakes 130, 135) each comprise a dense arrangement of small hooks, such as the hook portion of a Velcro® fastener. The bandage provides a suitable surface for the hooks to fasten onto.

In accordance with the invention, bandage 110 may be stored and packaged in a partially rolled configuration, as illustrated in FIG. 4. In an embodiment of the invention, bandage 110 is rolled by fully extending the bandage lengthwise such that the first or top surface with end brake 130 faces upward. The second end of bandage 110 with end brake 135 is then folded onto itself. The bandage 110 is then rolled from the second end towards the first end. During the rolling process, end brake 135 adheres to the bottom surface of bandage 110 while brakes 120 and end brake 130 adhere to the top surface of bandage 110. Of course, other methods of rolling the bandage, such as having end brake 130 fold back onto itself, are possible. In some embodiments after being rolled, bandage 110 may be vacuum sealed in a storage pouch.

In accordance with another embodiment of the invention, a wound dressing kit is provided comprising a prepackaged bandage and fastener system 100. As illustrated in FIG. 5, an exemplary prepackaged bandage comprises a sealed receptacle 150 having a wound pad 140 (FIG. 2) disposed therein. Sealed receptacle 150 is preferably made from a material that is durable enough to protect the contents during storage and yet suitable for use as an occlusion layer for a wound dressing. Suitable materials include foil and plastic. Wound pad 140 may comprise a generally non-abrasive, absorbent material such as treated gauze, e.g., hemostatic gauze, or untreated gauze. In some embodiments, an occlusive material may also be disposed in sealed receptacle 150.

In keeping with an aspect of the invention, the wound dressing kit may include means for attaching sealed receptacle 150 to bandage 110. An exemplary attachment means is a VELCRO® strip having an adhesive backing. The VELCRO® strip may be attached to sealed receptacle 150 by bringing the adhesive backing in contact with sealed receptacle 150. The user can then dress a wound by removing wound pad 140 from the sealed pouch, applying wound pad 140 to the wound and winding bandage 110 around the wound such that receptacle 150 covers wound pad 140. In this configuration, receptacle 150 actually functions as an occlusive layer for the wound dressing.

A method for applying the combined bandage and fastener system according to the present invention to a wound comprises unrolling the bandage 110 a user-selected amount to one of the plurality of brakes 120; applying the bandage to a wound; wrapping the wound with the bandage 110; and securing the bandage with at least one of the plurality of brakes or the at least one end brake.

The above-described invention is useful for the treatment and dressing of wounds by reducing the time required to treat and dress a wound. The invention is particularly useful in providing means for quickly controlling bleeding in situations where there are limited treatment materials or where time is vital.

Although the present invention has been described in terms of particular preferred and alternative embodiments, it is not limited to those embodiments. Alternative embodiments, examples, and modifications which would still be encompassed by the invention may be made by those skilled in the art, particularly in light of the foregoing teachings.

Those skilled in the art will appreciate that various adaptations and modifications of the preferred and alternative embodiments described above can be configured without departing from the scope and spirit of the invention. Therefore, it is to be understood that, within the scope of the appended claims, the invention may be practiced other than as specifically described herein.

We claim:

1. A combined bandage and fastener system, comprising:
a flexible bandage having a first end and a second end;
at least one end brake attached to a surface of said flexible bandage and disposed proximate to at least one of the first end or the second end of the bandage; and
a plurality of brakes attached to at least one surface of said flexible bandage between the first end and the second end of said flexible bandage,
wherein said plurality of brakes and said at least one end brake each comprise a strip of a dense arrangement of hooks.

2. The system of claim 1, wherein the brakes comprising said plurality of brakes are evenly spaced along the length of said flexible bandage.

3. The system of claim 1, comprising:
a first end brake attached to a first surface of said flexible bandage and disposed proximate to the first end; and
a second end brake attached to a second surface of said flexible bandage and disposed proximate to the second end.

4. The system of claim 3, wherein the first end brake and the second end brake each comprise a strip of a dense arrangement of hooks.

5. The system of claim 3, wherein the plurality of brakes are attached to the first surface of said flexible bandage.

6. The system of claim 3, wherein the plurality of brakes are attached to the second surface of said flexible bandage.

7. The system of claim 1, wherein said at least one end brake has a width that is greater than a width of said plurality of brakes.

8. The system of claim 1, wherein said plurality of brakes and said at least one end brake extend the width of said flexible bandage.

9. The system of claim 1, wherein said plurality of brakes extend along part of the length of said flexible bandage.

10. The system of claim 1, wherein said plurality of brakes are disposed at an angle to a lateral axis of said flexible bandage.

11. A combined bandage and fastener system, comprising:
an elastic bandage;
a plurality of brakes comprising strips of a dense arrangement of hooks, said strips spaced apart on a first surface of the elastic bandage and extending the width of the elastic bandage; and
at least one end fastener disposed at an end of the elastic bandage.

12. A method for packaging the system of claim 1, comprising:
extending said bandage lengthwise;
folding the second end of the bandage;
rolling said bandage from the second end towards the first end thus forming a rolled body; and
attaching the at least one end brake to the rolled body.

13. A method for packaging according to claim 12, further comprising vacuum packaging the rolled body.

14. A method of applying the combined bandage and fastener system of claim 1 to a wound, comprising:
unrolling the bandage a user-selected amount to one of the plurality of brakes;
applying the bandage to a wound;
wrapping the wound with the bandage; and
securing the bandage with at least one of the plurality of brakes or the at least one end brake.

15. A wound dressing kit, comprising:
a sealed receptacle comprising the combined bandage and fastener system of claim 1; and
a wound pad.
